(19) United States
(12) Patent Application Publication
(10) Pub. No.: US 2023/0157308 A1
(43) Pub. Date: May 25, 2023

(54) PERMEABILIZED MICROBIAL CELL CATALYSTS

(71) Applicant: DANMARKS TEKNISKE UNIVERSITET, Kongens Lyngby (DK)

(72) Inventors: Peter Ruhdal Jensen, Kongens Lyngby (DK); Christian Solem, Kongens Lyngby (DK); Jianming Liu, Kongens Lyngby (DK); Robin Dorau, Kongens Lyngby (DK); Qi Wang, Kongens Lyngby (DK)

(21) Appl. No.: 17/920,061

(22) PCT Filed: May 25, 2021
(86) PCT No.: PCT/EP2021/063835

§ 371 Date: Oct. 20, 2022

(30) Foreign Application Priority Data
May 25, 2020 (EP) 20176344.8

(51) Int. Cl. A23C 9/123; A23C 9/12; A23L 2/60; A23L 33/125

(52) U.S. Cl. CPC A23C 9/123; A23C 9/1206; A23L 2/60; A23L 33/125

(57) ABSTRACT

A permeabilized whole cell catalyst and method for conversion (reducing the amount) of a target substrate to one of more product are provided, wherein the catalyst is obtained by incubating the cells with a permeabilization agent selected from a fatty acid and fatty acid derivative, and wherein the permeabilized cells comprise an intracellular enzyme for catalyzing conversion of the target substrate. The target substrate transits through the channel, holes, or pore made in the cell membrane by the permeabilization agent, and is converted to products by enzymes retained within the cell.

### FIELD OF THE INVENTION

The present invention relates to use of permeabilized microbial cells as whole-cell catalysts for conversion (partial or full) of a target substrate(s) to one or more products, wherein the cells are permeabilized using a permeabilization agent selected from fatty acids and fatty acid derivatives, and wherein the cells comprise an enzyme capable of converting the substrate, and wherein the substrate can enter the permeabilized cells.

### BACKGROUND OF THE INVENTION

Many microbial enzymes with potential for industrial applications are retained within the microbial cells in which they are produced, which makes recovery and subsequent purification difficult.

Further, enzymes, while inside cells, are in a protected environment and are often more stable than when isolated. But for use of such enzyme(s) inside the cells, the substrate(s) must be able to cross the cell envelope to reach the enzyme(s), which may decrease the reaction rate obtained with such cells when compared to the corresponding isolated enzyme. One way to circumvent substrate transfer limitations involves the permeabilization of the cell wall and membranes by a chemical (e.g. by adding detergents or solvents) or physical (e.g. temperature shock) treatment. However, these procedures may interfere with the manufacturing and downstream processes, besides damaging the cells.

These problems are illustrated by the enzyme beta-galactosidase of Streptococcus thermophilus which, because of its food-grade classification, has attractive possibilities for application in the production of low-lactose foods. Since beta-galactosidase in S. thermophilus is a cytoplasmic enzyme, its isolation and use as a catalyst may only be achieved through the perturbation of cell integrity, either by sonic disruption or by chemical treatment such as using solvent mixtures. Sonication of the cells results in the release of beta-galactosidase, whereas permeabilization with organic solvents allows the passage of lactose to the cell interior while the beta-galactosidase is retained within the cell. Permeabilized cells may be used in place of purified beta-galactosidase in the production of low-lactose foods. Somkuti et al., 1996 found that several detergents and the commercial bile salt preparation Oxgall were effective in disrupting membrane structures in S. thermophilus to allow lactose influx but without causing enzyme leakage or denaturation. However, naturally a concern must be addressed in relation to the use of such chemicals in relation to food products, as residual permeabilizing agents may remain associated with the concentrated cell preparations intended for food applications (even after washing the cells).

The main concern when using solvent or detergent permeabilization of microbial cultures in the preparation of foods or beverages is solvent or detergent residues remaining associated with treated cells and ending up in finished food or beverage products. E.g. organic solvents such as toluene or acetone-toluene mixtures are excellent permeabilizing agents, but their residues would surely find objection when used in foods. In this regard, ethanol may be a better solvent choice, since it is already present in trace amounts in many fermented dairy foods consumed by humans. Somkuti et al., 1998 found that ethanol is efficient as a permeabilizing agent for increasing the level of measurable beta-galactosidase activity.

However, traces of any solvent, even ethanol, in a food or beverage, due to their treatment with permeabilized cells during manufacture, is considered undesirable and hence there remains a need to provide food grade microbial cell catalysts that avoid these problems.

### SUMMARY OF THE INVENTION

In a first aspect, the present invention provides a method for reducing the amount of a substrate in a sample, said method comprising the steps of:
a. providing microbial cells comprising at least one intracellular enzyme for catalyzing conversion of said substrate into one or more products,
b. incubating said microbial cells with a permeabilizing agent, wherein said permeabilization agent is a monoglyceride,
c. optionally harvesting permeabilized cells obtained in step (b),
d. incubating permeabilized cells obtained in step (b) or harvested cells obtained in step (c) with said sample comprising said substrate,
wherein said microbial cells are susceptible to permeabilization by said permeabilization agent,
wherein steps (a) and (b), and optionally step (c), are carried out prior to step (d), and
wherein said permeabilization facilitates enhanced import of the substrate by the permeabilized cells compared to non-treated cells.

A second aspect of the invention provides a whole-cell catalyst comprising permeabilized microbial cells obtained by incubating the cells with a permeabilization agent, wherein said permeabilization agent is a monoglyceride; wherein the permeabilized cells comprise at least one intracellular enzyme for catalyzing conversion of a target substrate; and wherein the permeabilized cells are in a frozen or dried state.

A third aspect of the invention provides a composition comprising
i. permeabilised microbial cells comprising at least one intracellular enzyme, said permeabilized cells obtained by incubating the cells with a permeabilization agent, wherein said permeabilization agent is a monoglyceride, and
ii. substrate(s) and product(s) of a reaction catalyzed by said at least one enzyme.

A forth aspect of the invention provides the use of permeabilized microbial cells comprising at least one intracellular enzyme as whole-cell catalyst in an enzyme reaction, wherein said permeabilized cells are obtained by incubating the cells with a permeabilization agent, wherein said permeabilization agent is a monoglyceride, and wherein permeabilization by said permeabilization agent facilitates enhanced import of substrate of said enzyme reaction by the permeabilized cells compared to non-treated cells.

### DESCRIPTION OF THE INVENTION

### Brief Description of the Figures

FIG. 1: Illustration of lactose hydrolysis by monolaurin-permeabilized cell.

FIG. 2: Lactose hydrolysis by S. thermophilus CS1980. Cells (100 mg/l) were permeabilized at 28° C. for 10 min using different concentrations of monolaurin. Hydrolysis was subsequently performed using 100 mg/l dry weight of permeabilized cells in a POM buffer solution comprising 5% lactose, incubated at 50° C.

FIG. 3: Acetoin production using monolaurin-permeabilized Lactococcus lactis biovar. diacetylactis RD1M5 cells. Cells (700 mg/l) were permeabilized at 30° C. for 20 min using 100 mg/L monolaurin. Acetoin was produced from 100 mM pyruvate solution. The experiments were carried out two times independently.

FIG. 4: Hydrolysis of lactose by S. thermophilus CS1980 where the cells have been permeabilized for 10 minutes at 28° C. using (i) 100 mg/ml monolaurin in POM buffer or (ii) 45% (v/v) aqueous ethanol, or (iii) have not been permeabilized (control). Hydrolysis of lactose in POM buffer (50 g/l lactose) at 50° C. was carried out at a cell concentration of approximately 100 mg/l.

FIG. 5: Reducing costs by growing the cell catalyst on mother liquor (ML). Different concentrations of ML (10, 20, 30, 40, 50%) with 10% HFI were used to grow the cell catalyst. The initial pH was adjusted to 7.0.

### ABBREVIATIONS, TERMS, AND DEFINITIONS

Fatty acid is a carboxylic acid with a long aliphatic chain, which is either saturated or unsaturated. Most naturally occurring fatty acids have an unbranched chain of an even number of carbon atoms, from 4 to 28. Fatty acids that are less than 6 carbon atoms long are defined as short-chain, while those with greater than 12 carbon atoms are long-chain fatty acids, and medium-chain fatty acids have between 6 and 12 carbon atoms.

Fatty acid derivative is an organic molecular entity derived from a fatty acid.

Monoglycerides are fatty acid derivatives composed of a molecule of glycerol linked to a fatty acid via an ester bond.

Permeabilizing agent is in the present invention selected from one or more fatty acid(s) and/or fatty acid derivative(s).

Permeabilized microbial cells of the present invention are cells which have been treated with the permeabilizing agent (fatty acid and/or fatty acid derivative), whereby channels, pores, holes or other means of passage have formed in the membrane through which a target substrate can transit, while the cells retain enzymes intracellularly that catalyze the conversion of the substrate. Permeabilized cells have enhanced import of the substrate compared to non-permeabilized cells.

Susceptible to permeabilization defines a property of a microbial cell that on interaction with the permeabilization agent (fatty acid and/or fatty acid derivative) results in formation of channels, pores, holes or other means of passage in its cell membrane through which target substrates can transit while enzymes which catalyze the conversion of the target substrates are retained within the cell.

Enhanced import of substrate refers to the enhanced passive intake of substrate by the permeabilized cells by the transit of the substrate through the channels, pores, holes or other means of passage formed in the cell membrane by the permeabilization agent.

Yoghurt starter bacterium refers to a bacterial preparation to assist the beginning of the fermentation process in preparation of yoghurt.

### DETAILED DESCRIPTION OF THE INVENTION

I. Permeabilized Microbial Cell Catalysts

The microbial cell according to the invention is a cell which is susceptible to permeabilization by fatty acids and/or fatty acid derivatives (as defined above)—in other words, a cell with which the fatty acid and/or fatty acid derivative interact to form channels, pores, holes or other means of passage in the cell's cell membrane. Examples of suitable fatty acids and/or fatty acid derivatives are given in section II.

The microbial cell of the invention is preferably a microorganism, e.g. a Gram-positive or Gram-negative bacterium, or a fungus (such as yeast). A non-exhaustive list of suitable candidate microorganisms is given as follows: a species belonging to the genus selected from among Escherichia, Streptococcus, Lactobacillus, Lactococcus, Lactovum, Pediococcus, Leuconostoc, Fructobacillus, Weissella, Oenococcus, Corynebacterium, Brevibacterium, Bacillus, Sporolactobacillus, Geobacillus, Halobacillus, Halolactibacillus, Tetragenococcus, Acetobacter, Acinetobacter, Proprionibacterium, Bifidobacterium, Kluyveromyces, Saccharomyces, Candida, and Aspergillus oryzae.

In one embodiment, the microbial cell of the invention is selected from lactic acid bacteria, such as from the group consisting of species of the genera Streptococcus, Lactobacillus, Lactococcus Abiotrophia, Aerococcus, Carnobacterium, Enterococcus, Leuconostoc, Oenococcus, Pediococcus, Tetragenococcus, Vagococcus, and Weissella. Even more preferably, in one embodiment, the microbial cell of the invention is selected from Streptococcus thermophilus, Lactobacillus casei, Lactobacillus plantarum, Lactobacillus helveticus, Lactobacillus delbrueckii, Lactobacillus acidophilus, and Lactococcus lactis.

While not wishing to be bound to theory, the action mechanism of the permeabilizing agent consists in a membrane-lytic behavior which stems from the amphipathic properties of fatty acids and fatty acid derivatives, leading to membrane destabilization and pore formation. The cells become “leaky” as a result of the treatment, resulting in release of e.g. essential cytoplasm components. The membrane-destabilizing activity causes increased cell permeability and cell lysis, leading to inhibition of bacterial cell growth (bacteriostatic action) or cell death (bactericidal action).

The present invention is based on the novel idea of exploiting the ability of fatty acids and fatty acid derivatives to make the cytoplasmic cell membrane of microbial cells permeable, thereby providing non-growing cells (non-viable) that can find a new use as whole cell catalysts.

The permeabilized microbial cell of the present invention is used as a permeabilized whole-cell catalyst, where substrate can easily enter the permeabilized cells (compared to non-treated cells), undergo enzymatic catalysis by a suitable enzyme within the cell, and products of the enzymatic reaction can then optionally also exit the cell. Small molecules thereby transit though the holes, pores, channels, or other means of passage made by the permeabilizing agent, while larger molecules (such as intracellular enzymes) are retained. The enzyme is thus kept within a stabilizing cell bag, which can easily be recovered from a solution by, for example, simple centrifugation.

II. Methods for Preparing Permeabilized Microbial Whole-Cell Catalysts

In preparing permeabilized cells of the present invention, microbial cells are simply brought in contact with the permeabilizing agent, such as fatty acids or fatty acid derivatives being added to a suspension of the microbial cells. The interaction of the fatty acid or fatty acid derivative with the cell membrane will lead to membrane destabilization and consequently its permeabilization.

The permeabilizing agent is selected from one of more fatty acid(s) or fatty acid derivative(s). In one embodiment the fatty acid or the fatty acid component of the fatty acid derivative is a medium chain fatty acid comprising between 6-12 carbon atoms. In another embodiment, the fatty acid or the fatty acid component of the fatty acid derivative is a long chain fatty acid comprising more than 12 carbon atoms. The fatty acid or the fatty acid component of the fatty acid derivative may be saturated or unsaturated, such as comprising one, two or more double bonds. The unsaturated fatty acids may be in the cis or trans form.

In one embodiment, the fatty acid is selected from one or more saturated fatty acids, such as caproic acid, caprylic acid, capric acid, lauric acid, palmitic acid, stearic acid, arachidonic acid, behenic acid, lignoceric acid, cerotic acid, myristic acid. In another embodiment, the fatty acid is selected from one or more unsaturated fatty acids, such as myristoleic acid, palmitoleic acid, sapienic acid, oleic acid, elaidic acid, vaccenic acid, linoleic acid, linoelaidic acid, linolenic acid, eicosapentaenoic acid, and arachidonic acid. In yet another embodiment, the fatty acid is a combination of saturated and unsaturated fatty acids, as disclosed herein. In a preferred embodiment, the fatty acid is lauric acid.

In one embodiment, the fatty acid derivative is selected from esters, ethers, alcohols, aldehydes, acetates, glycerides. In a preferred embodiment, the fatty acid derivative is selected from monoglycerides, such as monocaprin, monostearin, monolaurin, and monomyristate; preferably monolaurin and monomyristate.

In a preferred embodiment, the permeabilization agent is monolaurin.

The fatty acid or fatty acid derivatives for permeabilizing cells of the invention may be provided in purified form, such as commercially available fatty acids or fatty acid derivatives, e.g. obtainable from Sigma-Aldrich. Alternatively, the fatty acids may be provided as part of a natural composition, such as plant and vegetable oils.

In one embodiment, a single permeabilizing agent is used, selected from a fatty acid or a fatty acid derivative as disclosed herein. In another embodiment two or more permeabilization agents are used, selected from one or more fatty acids and/or one or more fatty acid derivatives as disclosed herein.

Permeabilization may be performed within a wide temperature range. It is preferred to avoid temperatures that are sufficiently high that they compromise cell and enzyme stability. Permeabilization temperature for mesophilic organisms is therefore preferably lower than 60° C., such as lower than 55° C., preferably even lower than 54, 53, 52, 51, of 50° C. Meanwhile for thermophilic organisms the permeabilization temperatures may be relatively higher, depending on the specific microorganism.

Without being bound by theory, the permeabilization reaction happens as soon as the cells are brought in contact with the permeabilization agent. In other words, in a well-mixed solution comprising microbial cells and the permeabilizing agent at saturation levels, the cells will quickly become permeabilized, and extended permeabilization reaction time is not needed. In one embodiment, the permeabilization time may be 5, 10, 15, 20, 25, 30, 35, 40, 45, 50, 55, or 60 seconds. In one embodiment, permeabilization time may be 1-60 minutes, such as 1-45 minutes, 1-30 minutes, 1-20 minutes; or even 1-10 minutes permeabilization time may suffice.

The amount/concentration of permeabilization agent applied for sufficient permeabilization of cells may depend on permeabilization conditions, such as the type of microbial cell to be permeabilized, concentration of cells, permeabilization time, and other medium conditions.

As an illustrative example, when 100 mg/l S. thermophilus cells are to be permeabilized: the examples provided herein show that 100 mg/l (=0.36 mM) monolaurin in POM buffer efficiently permeabilizes the cells within merely 10 minutes. Increasing the cell concentration may require increasing the concentration of permeabilizing agent to obtain efficient permeabilization within the same time period. Meanwhile, if the cell concentration is increased, but the concentration of permeabilizing agent is maintained, then the permeabilization time can be increased to obtain sufficient permeabilization. A person skilled in the art would know how to adjust/optimize these different parameters to obtain optimal permeabilization for a given organism at a desired cell concentration.

In one embodiment, the concentration of the permeabilization agent (fatty acid or fatty acid derivative) is 0.1-10 mM when permeabilizing a microbial sample having a cell concentration of 100 mg/l, such as 0.1-5 mM, such as 0.1-1 mM, such as 0.2-1 mM, such as 0.3-0.4 mM, preferably around 0.35 mM.

In a preferred embodiment, 0.1 g/l cells are treated with around 100 mg/l monolaurin for around 10 minutes.

After permeabilization-treatment, the cells may be used directly as permeabilized whole-cell catalysts, or they may preferably first be purified prior to use. A person skilled in the art would know how to perform such cell purification. Purification may simply be performed by pelleting the cells by centrifugation and removing the top liquid portion; or such as by simple filtration. The cells may further be washed, if needed, prior to their intended application. Any excess permeabilization agent left in the permeabilization medium may after harvesting the permeabilized cells be used in the subsequent permeabilization treatments of other cells.

In one embodiment, the permeabilized cells may be prepared as described above and further treated by a method ensuring preservation and possible storage of the cells prior to use. Methods of preservation, such as drying, freezing, or preparing liquid stocks of the microbial cells are known by a person skilled in the art and include, for example, glycerol stocks, or freezing concentrated cell slurries in liquid nitrogen, freeze-drying, spray drying, vacuum drying, etc.

One aspect of the present invention relates to a whole-cell catalyst comprising permeabilized microbial cells obtained by incubating the cells with a permeabilization agent selected from a fatty acid and fatty acid derivative; wherein the permeabilized cells comprise at least one intracellular enzyme for catalyzing conversion of a target substrate; and wherein the permeabilized cells are in a frozen or dried state.

Such whole-cell catalysts may be provided in bags, ampoules, tubes, vials, or the like depending on consumer preference.

The whole-cell catalyst may comprise any microbial cell susceptible to permeabilization by a permeabilization agent selected from a fatty acid and fatty acid derivative, as disclosed herein, said cell comprising an enzyme capable of catalyzing the conversion of a substrate, provided that (i) the size/shape/conformation of the enzyme ensures it being retained within the cell (not transiting though the channels, holes or pores created in the cell membrane by the permeabilization agent), and (ii) the size/shape/conformation of the substrate allows for the substrate to transit through channels, holes, or pores created in the cell membrane by the permeabilization agent.

In one embodiment, the whole-cell catalyst comprises frozen or dried preparations of permeabilized lactic acid bacteria, prepared as described herein, comprising beta-galactosidase EC 3.2.1.23 for catalyzing conversion of lactose to glucose and galactose.

In one embodiment, the whole-cell catalyst comprises frozen or dried preparations of permeabilized bacteria, prepared as described herein, comprising arabinose isomerase EC. 5.3.1.4 for isomerization of galactose to tagatose.

In one embodiment, the whole-cell catalyst comprises frozen or dried preparations of permeabilized bacteria, prepared as described herein, comprising xylose (glucose) isomerase EC 5.3.1.5 for isomerization of glucose to fructose.

In one embodiment, the whole-cell catalyst comprises frozen or dried preparations of permeabilized bacteria, prepared as described herein, comprising (i) alpha-acetolactate synthase EC 2.2.1.6 for conversion of pyruvate into alpha-acetolactate, and (ii) alpha-acetolactate decarboxylase EC 4.1.1.5 for conversion of alpha-acetolactate into acetoin.

III. Method for Reducing the Content of a Substrate in a Sample Using Permeabilized Microbial Cell Catalysts

One aspect of the present invention relates to a method for reducing the content of a substrate in a sample, said method comprising the steps of:
a. providing microbial cells comprising an intracellular enzyme for catalyzing conversion of said substrate into one or more products,
b. incubating said microbial cells with a permeabilizing agent selected from one or more fatty acid(s) and/or fatty acid derivative(s),
c. optionally harvesting permeabilized cells obtained in step (b),
d. incubating permeabilized cells obtained in step (b) or harvested cells obtained in step (c) with said sample comprising said substrate;
   wherein said microbial cells are susceptible to permeabilization by the fatty acid(s) or fatty acid derivative(s),
   wherein steps (a) and (b), and optionally step (c), are carried out prior to step (d), and
   wherein said permeabilization facilitates enhanced import of the substrate by the permeabilized cells compared to non-treated cells.

Microbial cells which have been permeabilized by treatment using fatty acid or fatty acid derivative are hereby used as a whole-cell catalyst. Examples of suitable fatty acids and/or fatty acid derivatives are given in section II. Whereas prior art has only used fatty acid and fatty acid derivative as antimicrobial agents as a means for killing cells—such as to avoid food spoilage by contaminating microbes, the present invention surprisingly reveals that such cells permeabilized by fatty acids or fatty acid derivatives are excellent whole-cell catalysts. The permeabilized cells function as a porous “bag” retaining and protecting the enzymes while substrates can freely transit though the cell membrane via the channels, holes or pores created by the action of the permeabilization agent, to be hydrolyzed within the cell. The products of the enzymes reaction may transit out through the cell membrane via the channels, holes or pores created by the action of the permeabilization agent, into the medium, or be consumed by the cell such as used by the cells metabolic machinery.

In the method of the present invention, for reducing the amount of substrate in a sample, the microbial cells are first permeabilized, and the permeabilized cells are then incubated with the sample comprising the substrate intended for catalysis by an intracellular enzyme of the cell. In other words, permeabilization is carried out prior to (separate from) the catalysis step where the permeabilized cells comprising intracellular enzymes are incubated with the relevant sample.

Depending on the application, it may be desirable to harvest the permeabilized cells prior to use to either be able to re-use excess permeabilization agent or to simply avoid having permeabilization agent carry-over in the catalytic reaction by the permeabilized cell catalysts. In other embodiments, the presence of permeabilization agent in the catalytic reaction is of no concern, and the permeabilized cells may be used directly in incubation with the sample comprising the substrate.

The high stability of permeabilized cells may in one embodiment support the re-use of the cells as cell catalyst. The cells may simply be harvested after use and re-used.

Suitable methods of harvesting may easily be identified by a person skilled in the art, such as centrifugation or other means of separating cells from a medium. This could have a significant impact on lowering the cost of use compared to e.g. conventional purified enzymes that cannot simply be collected and reused.

Numerous different applications exist for the method of the present invention. Any microbial cell susceptible to permeabilization by fatty acids or fatty acid derivatives may be used in the present method for conversion (reducing the amount) of a target substrate in a sample, provided that the cell comprises an (intracellular) enzyme capable of catalyzing the conversion of the substrate and that the size/shape/conformation of the substrate allows for the substrate to transit through channels, holes, or pores made in the cell membrane by the permeabilization agent, while the size/shape/conformation of the enzyme ensures it being retained within the cell (not transiting though the channels, holes or pores made in the cell membrane by the permeabilization agent).

In one embodiment, suitable applications of the present invention involves an enzyme catalyst having its smallest dimension being at least 1, 1.5, 2, 2.5, 3, 3.5, 4, 4.5, or 5 nm, for the enzyme to be retained within the cell, while the largest dimension of the substrate of the catalyst correspondingly does not exceed 1, 1.5, 2, 2.5, 3, 3.5, 4, 4.5, or 5 nm, for the substrate to be able to transit out of the permeabilized cell.

Embodiments of such applications of the invention may comprise hydrolysis of lactose by bacteria comprising beta-galactosidase; isomerization of galactose to tagatose using a bacterium comprising arabinose isomerase; isomerization of glucose to fructose using a bacterium comprising xylose (glucose) isomerase; conversion of pyruvate into alpha-acetolactate using a bacterium comprising alpha-acetolactate synthase; conversion of alpha-acetolactate into acetoin using a bacterium comprising alpha-acetolactate decarboxylase; etc. In one embodiment, the permeabilized whole cell catalyst of the invention may comprise more than one enzyme for a cascade reaction catalyzing the conversion of a substrate and its products to a final other product; such as, for example, a cell comprising both alpha-acetolactate synthase and alpha-acetolactate decarboxylase for conversion of pyruvate to acetoin.

The above applications are merely illustrative—as mentioned previously, the method has very broad application since the use of whole-cell catalysts of the invention for catalysis avoids the problems associated with enzyme secretion or purification. Furthermore, the enzymes in the whole cell catalysts of the invention are better protected and thus more active than secreted or isolated intracellular enzymes. Finally, the entry of substrate(s) into the cell and exit of product(s) from the cell is enhanced by the permeabilization and therefore sets the stage for more efficient catalysis.

The microorganisms applied in the method may naturally comprise the needed enzyme(s) for conversion of a target substrate, or they may be adapted or genetically modified by standard methods known in the art to express the required enzyme(s).

In a preferred embodiment, the application of the method of the present invention is within food industries, such as where the substrate of the invention is a food or beverage. Natural, non-GMO microbes are preferred for such application.

In one embodiment, the present method is suitable for reducing the lactose content of a dairy product—such as for producing lactose-reduced milk, butter milk, cream, butter, whey, yoghurt and yoghurt like products (e.g. Gaio®, Cultura®), junket, drink yoghurt, Skyr, Quark, Greek yoghurt, and other dairy products.

Specifically, such method of the present invention for reducing the lactose content of a dairy product comprises the steps of:
a. providing lactic acid bacteria comprising beta-galactosidase EC 3.2.1.23. for catalyzing conversion of lactose to galactose and glucose,
b. incubating said bacteria with a permeabilizing agent selected from one or more fatty acid(s) and/or fatty acid derivative(s),
c. optionally harvesting permeabilized bacteria obtained in step (b),
d. incubating permeabilized bacteria obtained in step (b) or (c) with said dairy product,
   wherein steps (a) and (b), and optionally step (c), are carried out prior to step (d)

Lactose is converted to glucose and galactose by the beta-galactosidase EC 3.2.1.23 enzyme. Several different bacteria naturally produce beta-galactosidase. When the method is used for reducing the lactose content in a dairy product, from a commercial/regulatory point of view, it is favorable to apply the method using a beta-galactosidase producing lactic acid bacterium, since lactic acid bacteria are naturally found in dairy products. In one embodiment, the lactic acid bacterium comprising beta-galactosidase EC 3.2.1.23 may be selected from Streptococcus thermophilus, Lactobacillus casei, Lactobacillus plantarum, Lactobacillus helveticus, Lactobacillus delbrueckii, Lactobacillus acidophilus, and Lactococcus lactis. Preferably, the lactic acid bacterium is a S. thermophilus strain.

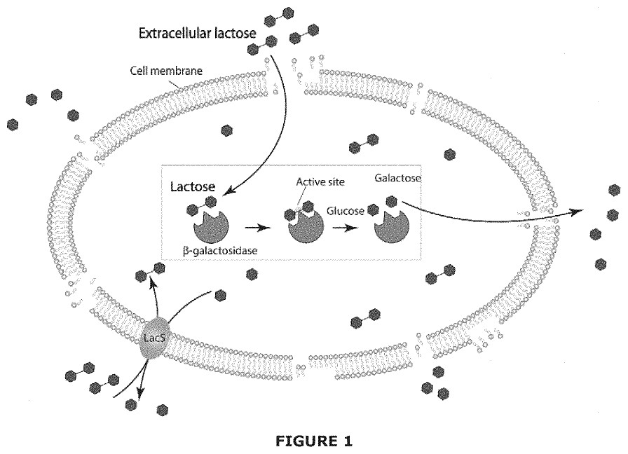

The lactic acid bacterium is incubated with the permeabilization agent under conditions easily optimized by a person skilled in the art by testing different combinations of temperature, time, cell concentration, permeabilization agent concentration etc. With their intended use in processing a dairy product, the permeabilized cells are preferably harvested after permeabilization-treatment, prior to adding them to the dairy product, and further optionally washed before adding them to the dairy product. Once added to the dairy product, the beta-galactosidase enzymes inside the permeabilized cells will facilitate hydrolysis of lactose in a dairy product into glucose and galactose, yielding a lactose-reduced dairy product (see FIG. 1).

In a one embodiment, the dairy product is a milk product, such as selected from skimmed milk, regular milk, whole milk, etc.

In one embodiment, the original lactose content of the dairy product is reduced by at least 50%, such as 55%, 60%, 65%, or even up to 70%. In one embodiment, 70% of the original lactose content of the dairy product is hydrolyzed by the permeabilized whole cell catalysts of the present invention comprising beta-galactosidase, such as 75%, 80%, 85%, 90%, or even 95% or above is hydrolyzed.

In another embodiment, the method of the present invention is suitable for preparing yoghurt (or yoghurt-like products) having a reduced lactose content. The method described above for preparing a dairy product having reduced lactose content may in this regard be followed by the addition of a step:
e. culturing yoghurt starter bacteria in the product obtained in step (d), wherein said starting dairy product is milk.

The added yoghurt starter bacteria will thereby facilitate conversion of the lactose-reduced milk to lactose-reduced yoghurt.

Specifically, the steps for making a yoghurt product having a low lactose content may be performed by the method of the present invention comprising the steps of:
a. providing lactic acid bacteria comprising beta-galactosidase EC 3.2.1.23 for catalyzing conversion of lactose to galactose and glucose,
b. incubating said bacteria with a permeabilizing agent selected from one or more fatty acid(s) and/or fatty acid derivative(s),
c. optionally harvesting permeabilized bacteria obtained in step (b),
d. incubating permeabilized bacteria obtained in step (b) or (c) with milk,
e. culturing yoghurt starter bacteria in the hydrolyzed milk product obtained in step (d).

Yoghurt starter bacteria are commonly known in the art, and may for example be selected from Streptococcus thermophilus and Lactobacillus delbruckii subsp. bulgaricus. In preparing lactose-reduced yoghurt, in one embodiment, the permeabilized cells comprising beta-galactosidase may hydrolyze between 50-100% of the lactose in milk, such as between 70-100% of the lactose. In another embodiment, the permeabilized cell catalyst may hydrolyze merely 50%, 55%, 60%, 65%, 70%, 75%, or 80% of the lactose prior to addition of the yoghurt starter culture, where after the remaining lactose content may be removed (utilized) by the yoghurt starter culture in combination with further hydrolysis by the permeabilized cell catalyst.

In a further embodiment, the method of the present invention may be applied to increase the sweetness of a lactose product. As a non-limiting example, partially hydrolyzed lactose may be used as a sweetener, as the presence of several types of sugars has a synergistic effect on sweetness. Thus the method can be used in all types of lactose-comprising products, such as yoghurts or even chocolates, to increase sweetness, and at the same time allow for reducing the overall added-sugar content.

In a further embodiment of the invention, value-added compounds may be produced. One such example is tagatose and fructose from the isomerization of galactose and glucose, respectively. This may be facilitated by permeabilized microbial cells comprising arabinose isomerase EC 5.3.1.4 (for conversion of galactose to tagatose) and/or xylose isomerase EC 5.3.1.5 (for conversion of glucose to fructose).

In one embodiment, the method of the invention comprises treating a microbial cell with a permeabilizing agent selected from one or more fatty acid(s) and/or one or more fatty acid derivative(s) as described herein (Section II), wherein the cell comprises (i) xylose isomerase EC 5.3.1.5 for conversion of glucose to fructose and/or (ii) arabinose isomerase EC 5.3.1.4 for conversion of galactose to tagatose; and wherein the substrate is glucose and/or galactose, respectively. Such cell for increasing sweetness of the milk product may be added to the dairy product in combination with the permeabilized microbial cell comprising beta-galactosidase previously described.

A microbial cell comprising arabinose isomerase EC. 5.3.1.4 and/or xylose isomerase EC 5.3.1.5 may in a preferred embodiment be selected from the group of lactic acid bacteria. These microbial cells may be added to a lactose-reduced milk product produced by the method of the invention. In a preferred embodiment, the microbial cell comprising arabinose isomerase EC 5.3.1.4 and/or xylose isomerase EC 5.3.1.5 is permeabilized, such as by methods described in section II for more efficient isomerization of the sugars.

In yet a further embodiment of the invention, the method may be applied to prepare a permeabilized whole-cell catalyst comprising alpha-acetolactate synthase EC 2.2.1.6 and/or alpha-acetolactate decarboxylase EC 4.1.1.5 to be used for efficient conversion of pyruvate into alpha-acetolactate and further acetoin, respectively—such as demonstrated in example 2.

IV. A Composition Comprising Permeabilized Microbial Cells

One aspect of the present invention relates to a composition comprising (i) permeabilised microbial cells comprising an intracellular enzyme, wherein the permeabilized cells are obtained by incubating the cells with a permeabilization agent selected from fatty acid and fatty acid derivative, and (ii) substrate(s) and product(s) of a reaction catalyzed by said enzyme—as defined in previous sections of the present application.

In one embodiment, the composition comprises (i) permeabilized lactic acid bacteria comprising beta-galactosidase EC 3.2.1.23, and (ii) a dairy product comprising one or more of lactose, glucose and galactose.

In another embodiment, the composition comprises (i) permeabilized lactic acid bacteria comprising beta-galactosidase and a permeabilized microbial cell comprising arabinose isomerase EC 5.3.1.4 and/or xylose isomerase EC 5.3.1.5 and (ii) a dairy product comprising one or more of lactose, glucose, galactose, tagatose, and sucrose.

V. Use of Permeabilized Cells as Whole-Cell Catalyst

One aspect of the present invention concerns the use of permeabilized microbial cells comprising an intracellular enzyme as whole-cell catalyst in an enzyme reaction wherein said permeabilized cells are obtained by incubating the cells with a permeabilization agent selected from a fatty acid and fatty acid derivative, and wherein permeabilization by said permeabilization agent facilitates enhanced import of substrate of said enzyme reaction by the permeabilized cells compared to non-treated cells. Examples of suitable fatty acids and/or fatty acid derivatives are given in section II. An essential prerequisite of the invention is that the substrate(s) of the enzyme reaction catalyzed by the intracellular enzyme of the permeabilized microbial cells has a size/shape/conformation that allows their transit through channel, holes or pores made in the cell membrane of microbial cells by the permeabilization agent.

Any given organism susceptible to permeabilization by fatty acids or fatty acid derivatives, comprising an intracellular enzyme having a substrate small enough to travel through the permeabilized cell membrane may be of use in the present invention—the enzyme and its substrate being defined in greater detail in previous sections of the present application.

In one embodiment, the permeabilized microbial whole cell catalysts of the present invention are lactic acid bacteria comprising beta-galactosidase for hydrolysis of lactose. The permeabilized cells are thereby used as whole-cell catalysts for lactose hydrolysis in e.g. dairy products. The beta-galactosidase of S. thermophilus or other lactic acid bacteria naturally contributes to the partial hydrolysis of lactose in dairy foods such as yogurt during product manufacture and again during the passage through the gastrointestinal tract, as the result of permeabilization by bile acids. Therefore, fortification of milk with permeabilized S. thermophilus prior to direct consumption or incorporation into milk-based products as a source of beta-galactosidase thereby provides a close to “natural process”, wherein the beta-galactosidase of inherently safe and edible S. thermophilus needs no further purification or isolation to qualify for food-grade status.

In a further embodiment, permeabilized cells of the present invention may be used as whole-cell catalyst in producing value-added compounds. One example is a permeabilized microbial cell capable of conversion of glucose and galactose to fructose and tagatose.

VI. A Method of Detecting Products Produced by the Permeabilized Cells

Methods for detecting and quantifying products, such as sugars, produced by a microbial cell of the invention include high performance liquid chromatography (HPLC) combined with refractive index detection to identify and quantify the products compared to standards, as one ordinary skilled in the art would be familiar with. Example 1 comprises the outline of one method of detection and quantification of sugars.

VII. Advantages and Commercial Application

Many fatty acids and fatty acid derivatives are natural compounds, abundant in nature. For example monolaurin is approved by the Joint Food and Agriculture Organization/World Health Organization (FAO/WHO) as a safe food additive, and is in the United States approved by the Food and Drug Administration as generally regarded as safe (GRAS) designation for use in processed cheeses.

Fatty acids and their derivatives—such as monolaurin—therefore represent an excellent choice as a permeabilizing agent—especially within the food industry as they are natural compounds, which is (or may likely be) food approved and easy to handle.

Using monolaurin-permeabilized cells as a substitute for commercial purified R-galactosidases has many advantages such as low cost, low resource utilization, and possibility for clean-label status of lactose-free dairy products. Also of importance, is that it provides a more natural process when compared to commercial lactase solutions, where lactases are often derived from GMO's (engineered microorganisms).

Somkuti et al. 1998, have previously demonstrated that ethanol-permeabilized lactic acid bacteria can be used as lactase, however, using ethanol has some obvious drawbacks, such as in terms of handling the ethanol, as large amounts of ethanol are involved and some ethanol could end up in the final product. Kosher/Halal status of dairy products is increasingly important, and using ethanol as a permeabilizing agent may raise issues in this respect, as small amounts of ethanol could be introduced, with the cells, into the product. Furthermore, it was demonstrated in Example 4 that ethanol compromises the stability of the whole-cell catalyst, hence more cells will be needed to achieve a satisfactory degree of hydrolysis within a certain time limit. Meanwhile, cells permeabilized by the method of the present invention (using monolaurin as permeabilization agent) showed a higher stability. Summarized, Example 4 clearly shows that monolaurin-treated cells perform better as whole-cell catalysts than ethanol-treated cells.

Further, the high stability of monolaurin-permeabilized cells supports the re-use of them as cell catalyst. The cells may simply be harvested after use and re-used, which could further lower the cost of use compared to e.g. conventional purified enzymes that cannot simply be collected and reused.

### EXAMPLES

### Example 1: Hydrolysis of Lactose Using Monolaurin-Permeabilized S. thermophilus Cells

Cell permeabilization provides a new and efficient way for hydrolyzing the lactose contained in dairy products like milk and yoghurt to make lactose-free dairy products. The method relies on safe lactic acid bacteria (LAB) that express beta-galactosidase activity intracellularly. The present example demonstrates that lactose can enter the monolaurin-permeabilized LAB cells at a faster rate compared to non-treated LAB cells.

1.1 Strains and Medium

Originating from a Danish yoghurt, Streptococcus thermophilus strain CS1980 was isolated on M17 medium (Sigma-Aldrich, Darmstadt, Germany) with lactose (LM17) (CS1980). S. thermophilus strains were grown at 37° C. in LM17 medium.

1.2 Cell Permeabilization Using Monolaurin

S. thermophilus strains were grown in 500-ml Erlenmeyer flasks containing 200 mL of medium at 37° C. for 16 h without agitation. 2 mL of an overnight culture was used as inoculum. Cells were collected by centrifugation at 10,000×g for 10 min at 4° C., washed once with sterile POM buffer (50 mM K₂HPO₄/KH₂PO₄, 1 mM MgCl₂, pH 7.4) and finally re-suspended in POM buffer to an optical density (OD600) of 0.3, corresponding to approximately 0.1 g/L (dry weight basis). For permeabilization, cell suspensions were dispensed into sterile tubes (1 mL per tube) and centrifuged in a microcentrifuge at 10,000×g for 5 min at 4° C. After decanting the supernatants, cell pellets were resuspended in 1 mL POM buffer containing different concentrations (10 mg/l and 100 mg/l) of monolaurin (Toronto Research Chemicals, Toronto, Canada), and were maintained for 10 min at 28° C. Non permeabilized cells in POM buffer were used as control. 100 mg/l of monomyristin was also tested as premeabilization agent.

1.3 Hydrolysis of Lactose in Lactose Buffer

The cells were resuspended in POM buffered lactose (50 g/L) solution. The hydrolysis was carried out at 50° C. for 2 h.

1.4 High-Performance Liquid Chromatography Analysis of Sugars

The concentration of lactose, glucose and galactose was determined on an Ultimate 3000 high-performance liquid chromatography (HPLC) system (Dionex) equipped with an Aminex HPX-87H column (Bio-Rad) and a Shodex RI-101 detector (Showa Denko K.K.). The column oven temperature was set to 60° C., and the mobile phase consisted of 5 mM H₂SO₄. The flow rate used was 0.5 mL/min.

1.4 Results

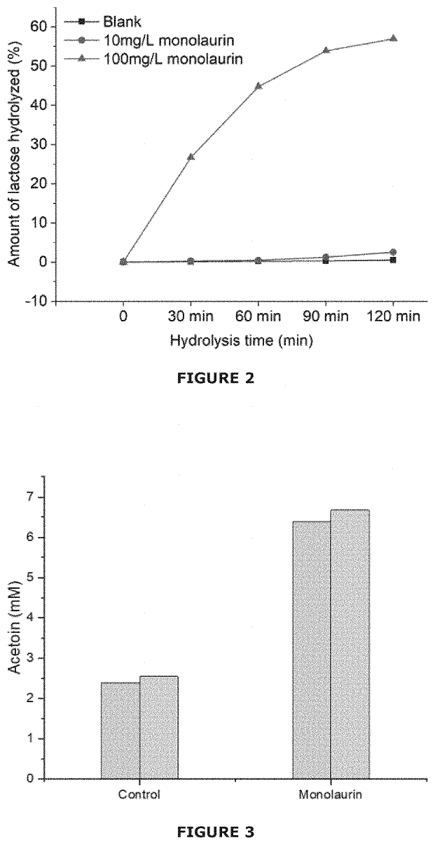

As shown in FIG. 2, when using monolaurin to permeabilize CS1980 cells (10 min at 28° C.), the resulting permeabilized cells were able to hydrolyze 55% of lactose in a 50 g/L solution in 2 hours at 50° C., using only 100 mg/L cells (dry weight basis). Hydrolysis results for non-treated cells (or cells treated with too low monolaurin concentration) was neglectable. Monomyristin (100 mg/L) was also tested and showed the same effectiveness in cell permeabilization as monolaurin.

### Example 2: Monolaurin-Permeabilized Cells Provides a New and Efficient Way for Converting Substrate into Valuable Biochemicals

Acetoin occurs naturally in many foods, in particular in dairy products where it contributes to the buttery aroma in yoghurt, cheese, butter and butter milk, and it is commonly added to various foods to enhance flavor. Microbial production of acetoin could provide a sustainable way. Strain RD1M5 used in the present example can produce acetoin from pyruvate. The present example demonstrates that monolaurin-permeabilized RD1M5 cells can produce acetoin more efficiently, as pyruvate influx to the cells is increased compared to non-treated cells.

2.1 Microorganisms and Media

Lactococcus lactis subsp. lactis biovar diacetylactis SD96 was kindly provide by Sacco S.r.l. (Cadorago, Italy). Adaptive laboratory evolution (ALE) was performed on strain SD96 in UHT (Ultra-High-Temperature processed) milk at high temperatures, whereby mutant strain RD01 was obtained. Further, by random mutagenesis using proflavine as the mutagen, the strains RD1M5 was derived from RD01.

2.2 Cell Permeabilization Using Monolaurin

Lactococcus lactis biovar. diacetylactis RD1M5 was permeabilized as described in example 1.2, using 100 mg/L monolaurin for 20 min at 30° C.

2.3 Acetoin Production

700 mg/L cells were suspended in POM buffer solution with 100 mM pyruvate. After 1 h, the cells were removed and the supernatant was used to quantify the acetoin concentration.

2.4 Analytical Methods

Quantification of lactose, lactate, acetoin, and citrate were carried out using an Ultimate high-performance liquid chromatography system equipped with a Aminex HPX-87H column (300×7.8 mm column) (Bio-Rad, Hercules, USA) and a Shodex RI-101 refractive index detector (Showa Denko K.K., Tokyo, Japan). The mobile phase was 5 mM H₂SO₄ with a flow rate of 0.5 mL/min and the column oven temperature was maintained at 60° C.

2.5 Results

FIG. 3 demonstrates that the monolaurin-permeabilized RD1M5 cells were able to produce much higher concentrations of acetoin from pyruvate in one hour compared to non-treated cells (control).

### Example 3: Monolaurin-Permeabilization is Efficient Over a Broad Temperature Range

3.1. Microbial Strains, Monolaurin-Permeabilization and Lactose Hydrolysis

S. thermophilus CS1980 is monolaurin-permeabilized using monolaurin in POM buffer at temperatures ranging 28 to 55° C. Controls are prepared without addition of monolaurin in the permeabilization step. Hydrolysis of lactose by monolaurin-treated cells is carried out in a POM buffered solution.

3.2. Results

A broad range of permeabilization temperatures: 20-50° C., are expected to be equally efficient. Temperatures as high as 50° C. are expected to work, but at higher temperatures, the cell stability (and likely also the enzyme stability, especially at the longer permeabilization time) may be compromised.

### Example 4: Monolaurin-Permeabilized Cells Perform Better than Ethanol-Permeabilized Cells

Somkuti et al. 1998, previously demonstrated that ethanol-permeabilized lactic acid bacteria can be used as a source of lactase activity. The present example compared the present invention to the current state of the art.

4.1. Microbial Strains, Permeabilization and Lactose Hydrolysis

S. thermophilus CS1980 was permeabilized for 10 minutes at 28° C. using (i) 100 μg/ml monolaurin in POM buffer or (ii) 45% (v/v) ethanol for 10 minutes, or (iii) not permeabilized (control). Hydrolysis of lactose in POM buffer (50 g/l lactose) at 50° C. was carried out at a cell concentration of approximately 100 mg/l.

4.2. Results

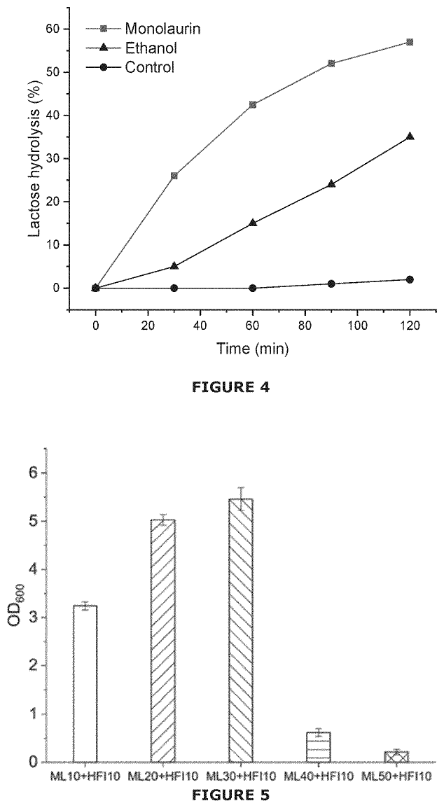

As seen in FIG. 4, monolaurin-permeabilized cells perform better than the control as well as the ethanol-permeabilized cells in terms of lactose conversion. Ethanol is generally known to affect the stability of enzymes, which is supported by the data in FIG. 4: it was found that ethanol compromised the stability of the whole-cell catalyst, which is unfavorable in terms of cost, as more cells will be needed to achieve a satisfactory degree of hydrolysis within a certain time limit.

### Example 5: Reducing Costs by Using Cheap Dairy Waste for Growing the Cell Catalyst

Instead of using expensive LM17 medium for growing the cell catalyst, it was explored whether it would be possible to use a dairy waste stream, i.e. whey mother liquor (ML) for growing the cells, in combination with hydrolyzed full whey protein liquid (HFI) as nitrogen source.

ML is a dairy waste stream produced after extraction of lactose from whey permeate; its composition is shown in Table 1. HFI is a hydrolyzed full whey protein hydrolysate manufactured; its composition is shown in Table 2.

TABLE 1
The composition of mother liquor (ML)^{a}

| | mM (mmol/L) |
|---|---|
| Lactose^{b} | 446.3 ± 12.4 |
| Citrate | 340.8 ± 4.7 |
| Lactate | 106.5 ± 2.6 |

^{a}The composition may vary from batch to batch.
^{b}Due to the low lactose solubility at low temperatures, it can crystallize in the fridge and therefore the concentration varies.

TABLE 2
The composition of HFI

| Unit (g/L) | Protein | Fat | Lactose | Salt |
|---|---|---|---|---|
| HFI | 238.3 ± 1.6 | 0.3 ± 0.1 | 5.8 ± 0.4 | 26.5 ± 2.1 |

Different concentrations of ML was tested, adding 10% HFI as a nitrogen source. Growth was followed simply by measuring optical density (OD₆₀₀). When using 30% ML and 10% HFI, OD₆₀₀ could reach 5.4, which is above what can be reached in LM17 (2% lactose) (FIG. 5). Higher concentrations of ML resulted in poorer growth, probably because of the high concentration of lactose and salts in ML. These results demonstrate that the cell catalyst can be cultivated on dairy waste streams and the low-cost for preparing the lactase cell catalyst shows its great potential for real-life applications.

Items of the Invention
1. A method for reducing the amount of a substrate in a sample, said method comprising the steps of:
   a. providing microbial cells comprising at least one intracellular enzyme for catalyzing conversion of said substrate into one or more products,
   b. incubating said microbial cells with a permeabilizing agent selected from among one or more fatty acid(s) and/or one or more fatty acid derivative(s),
   c. optionally harvesting permeabilized cells obtained in step (b),
   d. incubating permeabilized cells obtained in step (b) or harvested cells obtained in step (c) with said sample comprising said substrate,
   wherein said microbial cells are susceptible to permeabilization by the one or more fatty acid(s) and/or one or more fatty acid derivative(s), wherein steps (a) and (b), and optionally step (c), are carried out prior to step (d), and
   wherein said permeabilization facilitates enhanced import of the substrate by the permeabilized cells compared to non-treated cells.
2. The method according to item 1, wherein the one or more fatty acid permeabilizing agent(s) are selected from among caproic acid, caprylic acid, capric acid, lauric acid, palmitic acid, stearic acid, arachidonic acid, behenic acid, lignoceric acid, cerotic acid, myristic acid, myristoleic acid, palmitoleic acid, sapienic acid, oleic acid, elaidic acid, vaccenic acid, linoleic acid, linoelaidic acid, linolenic acid, eicosapentaenoic acid, and arachidonic acid.
3. The method according to any of item 1 or 2, wherein the one or more fatty acid derivative permeabilizing agent(s) are selected from among fatty acid esters, fatty acid alcohols, fatty acid aldehydes, fatty acid acetates, fatty acid glycerides, preferably monoglycerides, such as monocaprin, monolaurin, and monomyristate.

### REFERENCES

Somkuti, G. A., Dominiecki, M. E., Steinberg, D. H. 1996. Sensitivity of Streptococcus thermophilus to Chemical Permeabilization. Current Microbiology 32:101-105.
Somkuti, G. A., Dominiecki, M. E., Steinberg, D. H. 1998. Permeabilization of Streptococcus thermophilus and Lactobacillus delbrueckii subsp. bulgaricus with ethanol. Curr. Microbiol. 36(4):202-6.

1. A method for reducing the amount of a substrate in a sample, said method comprising the steps of:
a. providing microbial cells comprising at least one intracellular enzyme for catalyzing conversion of said substrate into one or more products,
b. incubating said microbial cells with a permeabilizing agent, wherein said permeabilization agent is a monoglyceride,
c. optionally harvesting permeabilized cells obtained in step (b),
d. incubating permeabilized cells obtained in step (b) or harvested cells obtained in step (c) with said sample comprising said substrate,
wherein said microbial cells are susceptible to permeabilization by said permeabilization agent,
wherein steps (a) and (b), and optionally step (c), are carried out prior to step (d), and
wherein said permeabilization facilitates enhanced import of the substrate by the permeabilized cells compared to non-treated cells.

2. The method according to claim 1, wherein the permeabilizing agent is selected from monolaurin and monomyristate.

3. The method according to claim 1, wherein said microbial cells provided in step (a) are bacteria selected from among Escherichia, Streptococcus, Lactobacillus, Lactococcus, Lactovum, Pediococcus, Leuconostoc, Fructobacillus, Weissella, Oenococcus, Corynebacterium, Brevibacterium, Bacillus, Sporolactobacillus, Geobacillus, Halobacillus, Halolactibacillus, Tetragenococcus, Acetobacter, Acinetobacter, Proprionibacterium, and Bifidobacterium.

4. The method according to claim 1, wherein the sample is a food or beverage.

5. The method according to claim 1, for reducing the lactose content of a dairy product, said method comprising the steps of:
a. providing cells of a lactic acid bacterium comprising intracellular beta-galactosidase EC 3.2.1.23. for catalyzing conversion of lactose to galactose and glucose,
b. incubating said cells of (a) with a permeabilizing agent, wherein said permeabilization agent is a monoglyceride,
c. optionally harvesting permeabilized cells obtained in step (b),
d. incubating permeabilized cells obtained in step (b) or (c) with said dairy product,
wherein steps (a) and (b), and optionally step (c), are carried out prior to step (d).

6. The method according to claim 5, wherein the lactic acid bacterium is selected from among Streptococcus thermophilus, Lactobacillus casei, Lactobacillus plantarum, Lactobacillus helveticus, Lactobacillus delbrueckii, Lactobacillus acidophilus, and Lactococcus lactis.

7. The method according to claim 5, wherein the dairy product is a milk product, such as skimmed milk, regular milk, whole milk, butter milk, cream, whey, butter, yoghurt, and yoghurt-like product selected from among junket, drink yoghurt, Skyr, Quark and Greek yoghurt.

8. The method according to claim 5, wherein the permeabilized cells are harvested in step (c); wherein the dairy product in step (d) is milk, and wherein said method further comprises the step of:
e. culturing cells of a yoghurt starter bacterium in the product obtained in step (d).

9. The method according to claim 8, wherein the yoghurt starter bacterium is Streptococcus thermophilus or Lactobacillus delbruckii subsp. bulgaricus.

10. The method according to claim 5, wherein in step (d) said dairy product is additionally incubated with cells of a microorganism comprising (i) xylose isomerase EC 5.3.1.5 for conversion of glucose to fructose, and/or (ii) arabinose isomerase EC 5.3.1.4 for conversion of galactose to tagatose, and wherein said second microbial cell is permeabilized using a second permeabilization agent prior to incubating in step (d), wherein said second permeabilization agent is a monoglyceride.

11. A whole-cell catalyst comprising permeabilized microbial cells obtained by incubating the cells with a permeabilization agent, wherein said permeabilization agent is a monoglyceride; wherein the permeabilized cells comprise at least one intracellular enzyme for catalyzing conversion of a target substrate; and wherein the permeabilized cells are in a frozen or dried state.

12. Use of permeabilized microbial cells comprising at least one intracellular enzyme as whole-cell catalyst in an enzyme reaction, wherein said permeabilized cells are obtained by incubating the cells with a permeabilization agent, wherein said permeabilization agent is a monoglyceride, and wherein permeabilization by said permeabilization agent facilitates enhanced import of substrate of said enzyme reaction by the permeabilized cells compared to non-treated cells.

13. The use according to claim 12 wherein said permeabilized microbial cells are bacteria comprising beta-galactosidase, and wherein said substrate is lactose.
